(19) United States
(12) Patent Application Publication
(10) Pub. No.: US 2008/0131945 A1
(43) Pub. Date: Jun. 5, 2008

(54) Method For Producing 3-Hydroxypropionaldehyde

(76) Inventors: Tetsuo Toraya, Okayama (JP); Takamasa Tobimatsu, Okayama (JP); Mamoru Yamanishi, Lincoln, NE (US); Kouichi Mori, Okayama (JP); Hideki Kajiura, Hyogo (JP); Seiki Yamada, Ibaraki (JP); Michio Yuzuki, Chiba (JP); Muneaki Azuma, Okayama (JP); Tetsuya Hara, Osaka (JP); Shinzo Yasuda, Ibaraki (JP)

Correspondence Address: OCCHIUTI ROHLICEK & TSAO, LLP, 10 FAWCETT STREET, CAMBRIDGE, MA, 02138, US

(21) Appl. No.: 10/573,718

(22) PCT Filed: Sep. 29, 2004
(86) PCT No.: PCT/JP04/14213

§ 371 Date: Nov. 28, 2006

(30) Foreign Application Priority Data
Sep. 29, 2003 (JP) 2003-337663

(51) Int. Cl. C12P 7/24; C12P 7/52

(52) U.S. Cl. 435/141; 435/147

(57) ABSTRACT

A method for producing 3-hydroxypropionaldehyde from glycerin in high conversion ratio is provided. The method is characterized by comprising a step of dehydrating glycerin using a microbial cell and/or a treated microbial cell containing diol dehydratase and/or glycerol dehydratase, and optionally diol dehydratase reactivating factor and/or glycerol dehydratase reactivating factor, under conditions so as to give a value (X/Y²) calculated by dividing a catalytic amount [X (U/g glycerin)] of diol dehydratase and/or glycerol dehydratase by square of glycerin concentration [Y (g/100 ml)] within a range of 10 to 8,000, to produce 3-hydroxypropionaldehyde.

### TECHNICAL FIELD

The present invention relates to a method for producing 3-hydroxypropionaldehyde and a method for producing 1,3-propanediol, 3-hydroxypropionic acid, acrolein, acrylic acid and an acrylic ester produced from 3-hydroxypropionaldehyde produced by the method. In particular, the present invention relates to a method which can efficiently produce 3-hydroxypropionaldehyde in high purity and a method which can efficiently produce 1,3-propanediol, 3-hydroxypropionic acid, acrolein, acrylic acid and an acrylic ester in high purity from the 3-hydroxypropionaldehyde.

### BACKGROUND ART

1,3-Propanediol is a useful compound having a wide range of applications, for example, as a monomer used for producing polyester and polyurethane, and a starting material for synthesizing a cyclic compound.

As a method for synthesizing 1,3-propanediol, both of a method by chemical synthesis and a method by fermentation have been well-known. As the former method, for example, a method for producing 1,3-propanediol via carbonylation of ethylene oxide using a rhodium catalyst (for example, U.S. Pat. No. 4,873,378, U.S. Pat. No. 873,379 and U.S. Pat. No. 4,935,554), and a method for producing 1,3-propanediol by reducing 3-hydroxypropionaldehyde (for example, U.S. Pat. No. 2,434,110) have been known.

However, the method by the chemical synthesis is not sufficient in conversion ratio and selectivity, and not favorable in the viewpoint of cost, because a purification process is required to remove a by-product. In addition, if 3-hydroxypropionaldehyde as a raw material contains a by-product, a secondary product might be further formed in the subsequent production process of 1,3-propanediol, and this could require a difficult purification, or cause discoloration or undesirable polymerization in products such as fibers in a production of textiles using this 1,3-propanediol as a raw material thereafter. For this reason, a content of by-product in 3-hydroxyprpionaldehyde to be used as a raw material for producing 1,3-propanediol is desirably as low as possible.

Further, the latter method is a method of producing 1,3-propanediol through the fermentation of glycerin or glucose using a strain producing 1,3-propanediol such as Citrobacter, Clostridium, Enterobacter, Ilyobacter, Klebsiella, Lactobacillus and Pelobacter, and the like. This method comprises two-step reaction consisting of a step of converting glycerin to 3-hydroxypropionaldehyde (3-HPA) and water with a dehydratase and a step of reducing the resultant 3-HPA to 1,3-propanediol with a NAD⁺-link-oxidoreductase. In addition, to improve yield of desired 1,3-propanediol, a method for producing 1,3-propanediol from glycerin using a recombinant microorganism has been disclosed (for example, WO 98/21339, WO 99/58686, U.S. Pat. No. 6,025,184 and WO 01/12833).

However, in the production method by the fermentation, it has been known that, to obtain NADH necessary for the latter reaction among the reactions, the two-step reaction and a reaction to form NADH by dehydrogenation to dihydroxyacetone occur simultaneously. By this reason, there is a problem that a conversion ratio from glycerin to 1,3-propanediol by the general fermentation becomes as low as around 50% resulting in an insufficient yield of 3-HPA. To solve this problem, production of 1,3-propanediol using a recombinant microorganism has been reported. However, even if such microorganism is used, since a reaction to form NADH needs to occur in addition to the reaction from glycerin to 3-HPA by the similar reason to the above, it is very difficult to attain a high conversion ratio. Further, a fermentation culture generally contains many by-products such as nutrients contained in the culture and microbial products. For this reason, the method using fermentation is also not preferable from an economical viewpoint, because a purification process becomes complicated much more than that by chemical synthesis. In addition to the problem, there is another problem that the method by the fermentation often uses an organic solvent such as cyclohexane in a purification process of the desired product, 1,3-propanediol, and further needs post-treatment of such organic solvent considering the environment.

On the other hand, 3-hydroxypropionaldehyde, which is an intermediate in the method, can be used also as an intermediate in the production of 3-hydroxypropionic acid, acrolein, acrylic acid and acrylic esters, as well as 1,3-propanediol as described above. Among these products, acrylic acid, for example, has a wide range of applications, and has been used as a copolymer for acrylic fiber, adhesives or agglutinant in emulsion, as well as coating materials, textile processing, leather, construction materials, and the like. Production method for acrylic acid has been conventionally known, and it is generally produced by a two-step gas phase catalytic oxidation, that is, from propylene to acrolein and from acrolein to acrylic acid. In this case, acrolein used as an intermediate material can be also produced via treatment of 3-hydroxypropionaldehyde under acidic conditions. Accordingly, to produce acrylic acid in high purity and at low cost, it is desirable that acrolein and further 3-hydroxypropionaldehyde are efficiently produced in high purity.

As a method for producing 3-hydroxypropionaldehyde (3-HPA), a method for producing 3-HPA from glycerol using a recombinant strain obtained by cloning of diol dehydratase and/or glycerol dehydratase in combination with diol dehydratase reactivating factor and/or glycerol dehydratase reactivating factor (for example, Journal of Bacteriology, Vol. 181, No. 13, pp. 4110-4113, 1999; The Journal of Biological Chemistry, Vol. 272, No. 51, pp. 32034-32041, 1997; Arch. Microbiol., 174:81-88 (2000); The Journal of Biological Chemistry, Vol. 274, No. 6, pp. 3372-3377, 1999), and a method for converting glycerin to 3-HPA by culturing Klebsiella pneumoniae in a glycerin-rich culture medium followed by suspending in a buffer containing semicarbazide and glycerin (Applied and Environmental Microbiology, Vol. 50, No. 6, pp. 1444-1450, 1985) have been reported. Among the methods, however, the method for producing 3-HPA from glycerin using a recombinant strain is, in any case, a method to scholastically study a behavior in an initial stage of the reaction, and specific reaction conditions of the conversion from the glycerin to 3-HPA need more study from the industrial viewpoint. Further, in the latter method, though a yield of around 83% at most can be attained due to an accumulation of 3-HPA facilitated by the presence of a semicarbazide, when 3-HPA is recovered in the presence of the semicarbazide, 3-HPA forms a complex with the semicarbazide as shown in the following reaction scheme, from which 3-HPA cannot be recovered again. Thus, the method cannot provide 3-HPA as a simple substance.

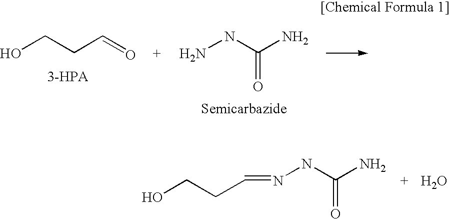

### DISCLOSURE OF THE INVENTION

### Problems to be Solved by the Invention

Thus, an object of the present invention is to provide specific conditions for producing 3-hydroxypropionaldehyde (3-HPA) in industrially high conversion ratio (that is, in high yield).

Another object of the present invention is to provide a method for producing 1,3-propanediol from the 3-HPA efficiently.

Further another object of the present invention is to provide a method for producing 3-hydroxypropionaldehyde acid from the 3-HPA efficiently.

Still further another object of the present invention is to provide a method for producing acrolein, acrylic acid or an acrylic ester from the 3-HPA in high purity and in high yield.

### Means to Solve the Problems

It has been generally well-known that activity (amount) of enzyme to act as a catalyst and concentration of substrate greatly influence conversion ratio. The present inventors have, after extensively studying a method for converting glycerin to 3-HPA to attain the objects in consideration of the circumstances, obtained such knowledge that there is a very strong correlation between catalytic activity and substrate concentration, in particular, in the conversion. Further, based on this knowledge, the present inventors have, after extensively studying a conversion from glycerin to 3-HPA using a microbial cell, toluene-treated microbial cell or immobilized microbial cell having diol dehydratase and/or glycerol dehydratase (herein collectively referred to as “diol/glycerol dehydratase” or simply as “dehydratase”) and diol dehydratase reactivating factor and/or glycerol dehydratase reactivating factor (herein collectively referred to as “diol/glycerol dehydratase reactivating factor” or simply as “dehydratase reactivating factor”), found that by applying an action of dehydratase with an amount of catalyst controlled so as to give a value (X/Y²) calculated by dividing a catalytic amount [X (U/g glycerin)] by square of glycerin concentration [Y (g/100 ml)] within a specified range, a conversion ratio in a high level corresponding to the industrial level, in particular, a conversion ratio not less than 70% can be attained, and 3-HPA can be produced in a high yield. Still further, the present inventors have also found that since almost all components other than glycerin such as nutrients are not used, particularly when a treated microbial cell is used, 3-hydroxypropionaldehyde can be easily produced in high purity only by removing the treated microbial cells by filtration or the like. Furthermore, the present inventors have obtained such knowledge that by hydrogenation of the thus obtained 3-hydroxypropionaldehyde, desired 1,3-propanediol can be produced in high purity and in high yield.

Further, the present inventors have also obtained such knowledge that acrolein can be obtained by reacting 3-hydroxypropionaldehyde scarcely containing by-products obtained as described above under acidic conditions, and this acrolein can be produced in high purity because the starting material scarcely contains any by-product, hence, from this acrolein, acrylic acid can be produced in high purity. In addition, the present inventors found that an acrylic ester can be also produced in high purity simply by the oxidative esterification of acrolein.

Namely, the object can be attained by a method for producing 3-hydroxypropionaldehyde which comprises a step of dehydrating glycerin using a microbial cell and/or a treated microbial cell containing diol dehydratase and/or glycerol dehydratase, and optionally diol dehydratase reactivating factor and/or glycerol dehydratase reactivating factor, under conditions so as to give a value (X/Y²) calculated by dividing a catalytic amount [X (U/g glycerin)] of diol dehydratase and/or glycerol dehydratase by square of glycerin concentration [Y (g/100 ml)] within a range of 10 to 8,000, to produce 3-hydroxypropionaldehyde.

The another object can be attained by a method for producing 1,3-propanediol comprising a step of hydrogenating 3-hydroxypropionaldehyde produced by the method to produce 1,3-propanediol.

The further another object can be attained by a method for producing 3-hydroxypropionic acid comprising a step of oxidizing 3-hydroxypropionaldehyde produced by the method to produce 3-hydroxypropionic acid.

The still further another object can be attained by a method for producing acrolein comprising a step of removing microbial cell and/or treated microbial cell from the reaction product containing 3-hydroxypropionaldehyde produced by the method, followed by reacting 3-hydroxypropionaldehyde under acidic conditions to produce acrolein; a method for producing acrylic acid comprising a step of oxidizing acrolein produced by the method to produce acrylic acid; and a method for producing an acrylic ester comprising a step of subjecting acrolein produced by the method to oxidative esterification to produce acrylic ester.

### BEST MODE FOR CARRYING OUT THE INVENTION

The present invention will be explained in detail below.

A first aspect of the present invention relates to a method for producing 3-hydroxypropionaldehyde which comprises a step of dehydrating glycerin using a microbial cell and/or a treated microbial cell containing diol dehydratase and/or glycerol dehydratase, and optionally diol dehydratase reactivating factor and/or glycerol dehydratase reactivating factor, under conditions so as to give a value (X/Y²) calculated by dividing a catalytic amount [X (U/g glycerin)] of diol dehydratase and/or glycerol dehydratase by square of glycerin concentration [Y (g/100 ml)] within a range of 10 to 8,000, to produce 3-hydroxypropionaldehyde. This means that among important factors considered to affect on a conversion ratio from glycerin to 3-HPA, in particular, an amount of catalyst greatly affects on a conversion ratio, and it has been revealed that by acting dehydratase on glycerin in such an amount that a value (X/Y²) calculated by dividing a catalytic amount [X (U/g glycerin)] of diol dehydratase and/or glycerol dehydratase by square of glycerin concentration [Y (g/100 ml)] is in a specified range as of 10 to 8,000, glycerin is selectively utilized in a conversion to 3-HPA without an occurrence of undesirable reaction such as a conversion of glycerin to NADH, and glycerin can be converted to 3-HPA in such a high conversion ratio as not less than 80%. Further, since conversion from glycerin to 3-HPA occurs preferentially and 3-hydroxypropionaldehyde to be produced scarcely contains a by-product, 3-hydroxypropionaldehyde can be produced in high purity by such a simple procedure as to separate/remove microbial cell/treated microbial cell.

In the present invention, a value (X/Y²) calculated by dividing a catalytic amount [X (U/g glycerin)] of diol dehydratase and/or glycerol dehydratase by square of glycerin concentration [Y (g/100 ml)] is in the range of 10 to 8,000. In this case, when X/Y² is fallen in this range, a high conversion ratio, for example, a conversion ratio of not less than 70% can be attained. On the contrary, when the X/Y² deviates from the range, the conversion ratio from glycerin to 3-HPA remarkably decreases. Lower limit of the X/Y² is preferably 7,000, 6,500, 5,500 and 5,000 in this order.

As used herein, “glycerin concentration [Y (g/100 ml)]” represents a weight of glycerin contained in 100 ml of a solution. For example, since 1 L of 50 mM potassium phosphate buffer (pH 8) containing 1 M of glycerin contains 92 g of glycerin, a glycerin concentration (Y) of this case becomes 9.2 (g/100 ml).

Further, as used herein, “catalytic amount [X (U/g glycerin)]” represents a value of a unit (U) of diol/glycerol dehydratase enzyme divided by a weight (g) of glycerin. In this case, “1 U” as a unit of enzyme means an ability of microbial cell and/or treated microbial cell exhibiting an activity of diol/glycerol dehydratase enzyme to convert 1 μmol of glycerin to 3-HPA per 1 minute, corresponds to an ability of microbial cell and/or treated microbial cell exhibiting a diol/glycerol dehydratase enzyme activity to convert 1 μmol of 1,2-propanediol to propionaldehyde per 1 minute. In this connection, in the present invention, the formations of propionaldehyde and 3-hydroxypropionaldehyde were detected by a detection method using 3-methyl-2-benzothiazolinonehydrazone hydrochloride. For example, when a toluene-treated microbial cell having an activity of 200 U is added to 1 L of 50 mM potassium phosphate buffer (pH 8) containing 1 M of glycerin, a catalytic amount (X) becomes 2.17 (U/g glycerin) (=200/92).

In the present invention, glycerin concentration (Y) is not especially limited, and may be selected, as appropriate, depending on viscosity of reaction liquid, amount of enzyme to be added, kind and intensity of enzymatic activity, concentration and purification processes after reaction. Lower limit of glycerin concentration (Y) is preferably not less than 0.5 g, more preferably not less than 1 g, and most preferably not less than 2 g, per 100 ml of solution. Also, upper limit of glycerin concentration (Y) is preferably not more than 60 g, more preferably not more than 50 g, and most preferably not more than 40 g, per 100 ml of solution. In this case, when glycerin concentration is less than 0.5 g/100 ml, due to too low glycerin amount in a solution, an amount of 3-HPA to be formed would become unduly low, and the concentration could be not economical from the industrial viewpoint. On the contrary, when glycerin concentration is over 60 g/100 ml, a viscosity of reaction liquid would increase thereby making homogeneous mixing with microbial cell/treated microbial cell difficult, glycerin could be impossible to efficiently receive an action of diol dehydratase/glycerol dehydratase.

In the present invention, dehydratase including diol dehydratase and glycerol dehydratase is coenzyme B12 dependent type as described above, and presence of coenzyme B12 is inevitable to convert glycerin to 3-HPA. Amount of coenzyme B12 to be present in the conversion from glycerin to 3-HPA is not especially limited so long as the conversion from glycerin to 3-HPA proceeds sufficiently thereby, and differs depending on concentration of substrate and the like. An amount of coenzyme B12 to be present is preferably in the range of 1 to 1,000 μM, more preferably 10 to 800 μM in a concentration of coenzyme B12 per 50 mM of substrate concentration. Further, amount of microbial cell and/or treated microbial cell is not especially limited so long as the catalytic amount of enzyme satisfies the relation: X/Y²=10 to 8,000, and a conversion of glycerin to 3-HPA sufficiently proceeds therewith, and differs depending on form of microbial cell and/or treated microbial cell to be used (microbial cell, immobilized microbial cell and immobilized factor) and source thereof, concentration of substrate (glycerin) and volume of reaction liquid. For example, microbial cell and/or treated microbial cell may be used in a batch type or in a flow reaction. When immobilized microbial cell is used in a flow reaction, an amount of microbial cell and/or treated microbial cell differs depending on kind of microorganism to be used, life of enzyme, flow rate of reaction liquid (LHSV) and the like. Also, an amount of microbial cell and/or treated microbial cell is not especially limited so long as the conversion of glycerin to 3-HPA sufficiently proceeds therewith. It usually about 10 to 100 times of amount as compared to that of batch type is used. Lower limit of catalytic amount (X) of enzyme, although be easily calculated from the value of X/Y², is preferably not less than 2.5 U/g glycerin, for example. Similarly, upper limit of catalytic amount (X) of enzyme can be also easily calculated from the value of X/Y², suitable value of Y and the like, but it is preferably not more than 27,000,000 U. In this case, when the catalytic amount of enzyme is less than 2.5 U/g glycerin, an action of enzyme to glycerin could be insufficient, and yield of desired 3-HPA could be also insufficient. On the contrary, even when a catalytic amount is over 28,800,000 U/g glycerin, effects obtained by amount of enzyme added could fail to be obtained which is not preferable from the economical viewpoint. In this connection, as for unit of the enzyme, an ability of microbial cell and/or treated microbial cell exhibiting diol/glycerol dehydratase enzymatic activity to convert 1 μmol of 1,2-propanediol to propionaldehyde is defined as “1 U”, and measuring method thereof is not especially limited, and any method can be used so long as formation of propionaldehyde from 1,2-propanediol can be detected thereby. In the present invention, formations of propionaldehyde and 3-hydroxypropionaldehyde were detected by a method to detect using 3-methyl-2-benzothiazolinone hydrochloride.

In the present invention, “diol/glycerol dehydratase” or “dehydratase” is an enzyme having a catalytic action to convert glycerin to 3-hydroxypropionaldehyde (herein also referred to as “3-HPA”) and water by dehydration. Any known enzyme can be used without special limitation so long as the enzyme has such an action. Among these enzymes, in view of life of enzyme, glycerol dehydratase is preferably used.

In the present invention, glycerol dehydratase is not especially limited, and glycerol dehydratase derived from any source having/expressing this enzyme may be used. Specifically, these include microorganisms belonging to Klebsiella genus, Citrobacter genus, Clostridium genus, Lactobacillus genus, Enterobacter genus, Caloramator genus, Salmonella genus and Listeria genus, and more specifically, glycerol dehydratase derived from Klebsiella pneumoniae, Citrobacter pneumoniae, Clostridium Pasteurianum, Lactobacillus leichmannii, Citrobacter intermedium, Lactobacillus reuteri, Lactobacillus buchneri, Lactobacillus brevis, Enterobacter agglomerans, Clostridium butyricum, Caloramator viterbensis, Lactobacillus collinoides, Lactobacillus hilgardii, Salmonella typhimurium, Listeria monocytogenes and Listeria innocua. These glycerol dehydratases may be used alone or in combination of two or more kinds, or may be used in combination with diol dehydratase described in detail below.

Method for isolating glycerol dehydratase from the sources is not especially limited, and glycerol dehydratase may be isolated from any microorganism as described above similarly to a known separation and isolation method such as extraction and column chromatography.

Further, in the present invention, diol dehydratase is also not especially limited. Any diol dehydratase derived from any source having/expressing this enzyme may be used. Specifically, these include microorganisms belonging to Klebsiella genus, Propionibacterium genus, Clostridium genus, Lactobacillus genus, Salmonella genus, Citrobacter genus, Flavobacterium genus, Acetobacterium genus, Brucella genus and Fusobacterium genus, and more specifically, diol dehydratase derived from Klebsiella pneumoniae, Propionibacterium freudenreichii, Clostridium glycolicum, Lactobacillus brevis, Salmonella typhimurium, Citrobacter freundii, Lactobacillus buchneri, Brucella melitensis, Fusobacterium nucleatum, Klebsiella oxytoca, Salmonella typhimurium, Listeria monocytogenes and Listeria innocua. These diol dehydratases may be used alone or in combination of two or more kinds, or may be used in combination with glycerol dehydratase described in detail above.

Method for isolating diol dehydratase from the sources is not especially limited, and diol dehydratase may be isolated from any microorganism as described above similarly to a known separation and isolation method of enzyme such as extraction and column chromatography.

Microbial cell and/or treated microbial cell may contain, in addition to diol/glycerol dehydratase, diol/glycerol dehydratase reactivating factor. In this case, in view of conversion ratio of glycerin to 3-HPA and reactivation of enzyme, microbial cell/treated microbial cell having diol/glycerol dehydratase reactivating factor is preferably used.

In the present invention, “diol/glycerol dehydratase reactivating factor” or “dehydratase reactivating factor” is referred to as a factor to induce again (reactivate) an activity of dehydratase, which has been deactivated by catalyzing a reaction of glycerin to 3-HPA+H₂O. In more detail, the coenzyme B12 is involved in the reaction of glycerin to 3-HPA+H₂O, which is catalyzed by dehydratase, and active center of the dehydratase is deactivated after developing and catalyzing the reaction of glycerin to 3-HPA+H₂O due to collapse of the coenzyme B12. Here, the dehydratase reactivating factor replaces the collapsed coenzyme B12 with a new coenzyme B12 to induce again (reactivate) an activity of the dehydratase and make the dehydratase reusable for dehydration reaction of glycerin. Thus, in the present invention, a factor having an ability to reactivate the dehydratase is referred to as “diol/glycerol dehydratase reactivating factor” or “dehydratase reactivating factor”. The dehydratase reactivating factor is not especially limited so long as the factor has an ability as described above, and well-known glycerol dehydratase reactivating factor which activates deactivated glycerol dehydratase and diol dehydratase reactivating factor which activates a deactivated diol dehydratase can be used. In this connection, dehydratase and dehydratase reactivating factor may be used in any combination of those described above. Dehydratase reactivating factor is preferably composed of a large subunit of diol dehydratase reactivating factor and/or glycerol dehydratase reactivating factor and a small subunit of diol dehydratase reactivating factor and/or glycerol dehydratase reactivating factor, more preferably composed of a large subunit of diol dehydratase reactivating factor and a small subunit of diol dehydratase reactivating factor and/or glycerol dehydratase reactivating factor, and particularly preferably composed of a large subunit of diol dehydratase reactivating factor and a small subunit of diol dehydratase reactivating factor. In this case, a large subunit of diol dehydratase reactivating factor includes, for example, ddrA (NCBI No. AF017781) and a large subunit of glycerol dehydratase reactivating factor includes, for example, gdrA (NCBI No. U30903), but not limited to these. A small subunit of diol dehydratase reactivating factor includes, for example, ddrB (NCBI No. AF017781) and a small subunit of glycerol dehydratase reactivating factor includes, for example, gdrB (NCBI No. U30903), but not limited to these. These reactivating factors may be used alone or as a mixture of two or more kinds. Further, order of a large subunit and a small subunit is not especially limited, and may be in any order of a large subunit and a small subunit, or a small subunit and a large subunit, or, when each of these units exists in plural, these units may exist in any of block or random, but from the viewpoint of a high reactivation ability, preferably a large subunit exist in upstream of a small subunit.

In the present invention, a source of the glycerol dehydratase reactivating factor is not especially limited. The factor is coded on a genome of a microorganism having glycerol dehydratase as described above, and composed of a large subunit and a small subunit. Specifically, glycerol dehydratase reactivating factors derived from microorganisms belonging to Lactobacillus genus, Klebsiella genus, Citrobacter genus, Clostridium genus and Enterobacter genus, and more specifically, the microorganisms include, for example, Lactobacillus sp., Klebsiella pneumoniae, Lactobacillus leichmannii, Citrobacter freundii, Citrobacter intermedium, Lactobacillus reuteri, Lactobacillus buchneri, Lactobacillus brevis, Clostridium Pasteurianum, Enterobacter agglomerans and Clostridium butyricum may be cited. Among these microorganisms, microorganisms belonging to Lactobacillus genus, for example, Lactobacillus sp., Lactobacillus leichmannii, Lactobacillus reuteri, Lactobacillus buchneri and Lactobacillus brevis may be preferably used, and glycerol dehydratase reactivating factors derived from Lactobacillus sp. and Lactobacillus reuteri are particularly preferably used. In this connection, sources of glycerol dehydratase and glycerol dehydratase reactivating factor may be the same or different from each other.

Method for isolating glycerol dehydratase reactivating factor from the sources is not especially limited, and glycerol dehydratase reactivating factor may be isolated from any microorganism as described above similarly to a well-known separation and isolation method of enzyme such as extraction and column chromatography.

Further, in the present invention, source of the diol dehydratase reactivating factor is not especially limited. The factor is coded on a genome of a microorganism having diol dehydratase as described above, and composed of a large subunit and a small subunit. In this connection, this glycerol dehydratase reactivating factor is preferably used because the factor can reactivate both of glycerol dehydratase and diol dehydratase. Specifically, diol dehydratase reactivating factors derived from microorganisms belonging to Klebsiella genus, Citrobacter genus, Propionibacterium genus, Lactobacillus genus, Flavobacterium genus and Acetobacterium genus may be used. More specifically, the microorganisms include, for example, Klebsiella pneumoniae, Citrobacter freundii, Propionibacterium freudenreichii, Lactobacillus brevis, Lactobacillus buchneri, Flavobacterium sp. and Acetobacterium sp. Among these microorganisms, diol dehydratase reactivating factors derived from microorganisms belonging to Lactobacillus genus, that is, Lactobacillus brevis and Lactobacillus buchneri, in particular, Lactobacillus brevis can be preferably used. In this connection, sources of diol dehydratase and diol dehydratase reactivating factor may be the same or different from each other, but from the viewpoint of more superior reactivation ability, a large subunit of diol dehydratase reactivating factor is preferably used. Accordingly, the combination of a large subunit of diol dehydratase reactivating factor and a small subunit of glycerol dehydratase reactivating factor and/or diol dehydratase reactivating factor is more preferably used.

Method for isolating diol dehydratase reactivating factor from the sources is not especially limited, and glycerol dehydratase reactivating factor may be isolated from any microorganism as described above similarly to a well-known separation and isolation method of enzyme such as extraction and column chromatography.

Alternatively, a recombinant microorganism containing a gene coding for dehydratase and/or dehydratase reactivating factor and a microorganism mutated so as to improve an activity of dehydratase and/or dehydratase reactivating factor may be used as a microorganism source having/expressing the dehydratase and/or dehydratase reactivating factor. These recombinant microorganisms can be prepared using a conventional method. For example, microorganisms expressing dehydratase include, specifically, those disclosed in Journal of Bacteriology, Vol. 181, No. 13, pp. 4110-411.3, 1999; The Journal of Biological Chemistry, Vol. 272, No. 51, pp. 32034-32041, 1997; Arch. Microbiol., 174:81-88 (2000); The Journal of Biological Chemistry, Vol. 274, No. 6, pp. 3372-3377, 1999, all of which were described in the section of Description of Related Art, as well as those disclosed in WO 98/21339, WO 99/58686, WO 98/21341, U.S. Pat. No. 6,025,184, WO 01/12833, WO 96/35795, WO 01/04324, FEMS Microbiology Letters 164 (1998) 21-28, Applied and Environmental Microbiology, January 1998, p. 98-105, Applied and Environmental Microbiology, December 1991, p. 3541-3546, and the like. Also, microorganisms mutated so as to improve an activity of dehydratase and/or dehydratase reactivating factor can be prepared using a conventional method, and include, specifically, for example, those disclosed in WO 00/70057 and the like.

In the present invention, 3-hydroxypropionaldehyde can be produced by dehydration of glycerin using microbial cell and/or treated microbial cell containing diol/glycerol dehydratase and, if necessary, diol/glycerol dehydratase reactivating factor. In this case, from the viewpoints that a very high conversion ratio can be attained as described later in detail, and 3-hydroxypropionaldehyde obtained contains less amount of by-products, preferably “microbial cell” and “treated microbial cell” do not use glycerin as an energy source of the microorganism. In the present invention, treated microbial cell is more preferably used. This is because use of treated microbial cell allows conversion of glycerin to 3-HPA by a method other than fermentation and thus attainment of high conversion ratio and low content of by-product.

In this connection, in the present invention, “microbial cell” is not especially limited so long as the microbial cell has diol dehydratase and/or glycerol dehydratase, and, if necessary, diol dehydratase reactivating factor and/or glycerol dehydratase reactivating factor, and an ability to convert glycerin to 3-HPA. It does not matter whether a microbial cell has a proliferating ability or not when the microbial cell is returned to a suitable culture medium after completion of the reaction of the present invention. Preferably, microbial cell is one in which 3-HPA is not further used to another reaction, and which allows conversion of glycerin to 3-HPA in a reaction system containing glycerin by a method other than fermentation, that is, does not assimilate glycerin nor proliferate using glycerin as an energy source. For example, these microbial cells include preferably a microbial cell obtained by culturing a microbial cell having an ability to convert glycerin to 3-HPA under aerobic conditions; a microbial cell obtained by culturing the microbial cell under conditions so as not to produce NADH and/or NADPH; and a recombinant microbial cell obtained by introducing diol/glycerol dehydratase or diol/glycerol dehydratase reactivating factor to a suitable host, and more preferably a microbial cell which has been genetically manipulated so as to have a plurality of copies of diol/glycerol dehydratase to highly express these enzymes and the like. Further, “fermentation” means an activity of microorganism performing, in a reaction system in which 3-hydroxypropionaldehyde is obtained from glycerin, at a same time, conversion of glycerin and/or proliferation using glycerin as an energy source and/or oxidation of glycerin and the like. Hence, in the present invention, “method other than fermentation” means a reaction method in which 3-hydroxypropionaldehyde is formed by dehydration of glycerin in a reaction system containing glycerin of the present invention, but at the same time not being accompanied by conversion of glycerin, proliferation using glycerin as an energy and oxidation of glycerin.

Herein, “treated microbial cell” means a microbial cell which has been treated so as to be easily used for the reaction of the present invention. Specifically, examples of the treated microbial cell include, for example, immobilized substance (for example, immobilized enzyme) such as immobilized dehydratase or immobilized dehydratase reactivating factor as described above; toluene-treated microbial cell which is obtained by treating microbial cell containing dehydratase or dehydratase reactivating factor as described above with an organic solvent such as toluene; and immobilized microbial cell of a microbial cell having dehydratase or dehydratase reactivating factor as described above. Among these, toluene-treated microbial cell and immobilized microbial cell are preferably used, and toluene-treated microbial cell is particularly preferable. Immobilizing method in producing a immobilized factor is not especially limited, and well-known methods such as a method by which a factor is immobilized on an insoluble substrate via covalent bond, ionic bond, adsorption or the like; a method by which a factor is cross-linked each other; and a method by which a factor is included in a network structure of a polymer, may be used.

Also, immobilizing method for producing an immobilized microbial cell is not especially limited, and conventional methods such as a method by which microorganism having dehydratase or dehydratase reactivating factor as described above is supported on an insoluble substrate; a method by which the microorganism is immobilized by being absorbed or included in a gel matrix; and a method by which the microorganism is entrapped in an internal space, may be used.

Further, production method for the toluene-treated microbial cell is also not especially limited. Conventional methods such as a method which comprises adding toluene to a microorganism having dehydratase or dehydratase reactivating factor as described above and stirring the mixture, thereby forming holes having such a size that an enzyme and a factor can not go out from the microbial cell may be used. In the method, though toluene was used to produce a toluene-treated microbial cell, another organic solvent such as acetone, hexane and ethyl acetate may be used instead of toluene. Among these, toluene is preferably used. In this case, amount of the organic solvent to be added is preferably in the range of 0.1 to 10% by mass, and more preferably 0.2 to 5% by mass. Further, the toluene-treated microbial cell is not especially limited, and can be prepared by conventional methods. For example, the following method can be used: A microbial cells cultured under suitable conditions are suspended in a buffer, and toluene is added to this suspension so as to give an adequate concentration, preferably a final concentration of 1% (v/v), then the suspension is vigorously stirred for a prescribed time, preferably about 5 minutes using a vortex mixer or the like to perform toluene treatment, subsequently the microbial cell after the toluene treatment is washed with a buffer. In the method, buffer is not especially limited, and well-known buffers can be used. 50 mM potassium phosphate buffer (25 ml of 1 M K₂HPO₄ and 100 ml of 1 M K₂HPO₄ are mixed, pH is adjusted at 8, then water is added to 25 ml of the solution to make 500 ml as a whole) is particularly preferably used.

In the present invention, immobilized microbial cell and immobilized factor may be used in any form, and specifically, used, for example, in a form of membrane-like, particulate, ribbon-like and folded layer-like. In view of easiness in handling, ribbon-like and particle-like are preferably used.

By using microbial cell and/or treated microbial cell, stabilization of microbial cell and/or treated microbial cell containing dehydratase and dehydratase reactivating factor can be performed. In particular, when treated microbial cell is used, in addition to an advantage that these microbial cell can be continuously and repeatedly used, another advantage exists that purity and yield of the desired 3-hydroxypropionaldehyde can be improved because glycerin can be conveniently converted to 3-hydroxypropionaldehyde without using a culture medium.

In the present invention, method for converting glycerin to 3-HPA is not especially limited, and a method similar to conventionally known methods can be used using a factor, an immobilized microbial cell and an immobilized factor. Differing from the case of fermentation, since the conversion of glycerin to 3-HPA according to the present invention is carried out using microbial cell and/or treated microbial cell (for example, toluene-treated microbial cell and immobilized microbial cell), 3-hydroxypropionaldehyde to be obtained scarcely contains by-product, and further, since almost all of glycerin is used for the reaction, a high conversion ratio can be attained. Further, in comparing with conventional chemical method, since raw material is mainly glycerin, and both the conversion ratio and selectivity to 3-hydroxypropionaldehyde are very high and purity is also high, a purification process to remove by-product can be performed conveniently, and the method is very advantageous from the economical viewpoint.

A preferable embodiment of the conversion of glycerin to 3-HPA according to the present invention will be described below. For example, when a particulate immobilized microbial cell and/or immobilized factor is used, it is added into a mixed solution containing a suitable buffer (for example, potassium phosphate buffer), an appropriate amount of coenzyme B12 as described above and glycerin, so that a enzyme as described above is suitably present, and then the resultant mixture is stirred at 10 to 90° C., preferably at 15 to 85° C. for 1 to 360 minutes, preferably for 5 to 120 minutes to form 3-HPA. The 3-HPA thus formed is in a mixed state with immobilized microbial cell on the particle, and can be easily separated from the immobilized microbial cell by a conventional method such as filtration, ultrafiltration and settling. Alternatively, 3-HPA may be formed by passing a mixed solution containing a suitable buffer (for example, potassium phosphate buffer), an appropriate amount of coenzyme B12 as described above, glycerin and the like through a column packed with a particulate immobilized microbial cell and/or immobilized factor at 10 to 90° C., preferably at 15 to 85° C., and at a flow rate of 0.1 to 50 LHSV, preferably 0.2 to 40 LHSV. In this case, glycerin concentration is not especially limited, so long as the factor can sufficiently act thereon, and it is varied with a catalytic amount of diol dehydratase and/or glycerol dehydratase, and also may be such a concentration at which X/Y² is not fallen within a range of 10 to 8,000. Preferably, it is in the range of 0.1 to 50% (w/v), more preferably 0.2 to 40% (w/v). Further, in the method, liquid used for dissolving glycerin is not especially limited so long as glycerin can be dissolved. Water, and various buffers such as physiological saline, potassium phosphate buffer, potassium citrate buffer, phosphate buffer, Good's buffer and Tris buffer may be used, for example. Among these, water, potassium phosphate buffer and phosphate buffer are preferably used. In the present invention, presence of potassium ion in a reaction system is preferable in view of an activity of enzyme. For the presence of potassium ion in the reaction system, buffers containing potassium salts such as potassium phosphate buffer, potassium citrate buffer and other potassium salt aqueous solution is particularly preferably used as a reaction medium. Concentration of potassium ion is not especially limited, but preferably in the range of. 5 mM to 1 M, and more preferably 10 to 500 mM.

According to the method of the present invention, the 3-HPA thus obtained is, after microbial cell and/or treated microbial cell being removed, hydrogenated to form desired 1,3-propanediol. Thus, a second aspect of the present invention relates to a method for producing 1,3-propanediol which comprises a step of removing the microbial cell and/or treated microbial cell from the 3-hydroxypropionaldehyde produced by the method of the present invention, subsequently hydrogenating said 3-hydroxypropionaldehyde to produce 1,3-propanediol. According to the method of the present invention, since the conversion from glycerin to 3-HPA occurs preferentially in a high conversion ratio, 3-propionaldehyde of the product can be produced in a high purity without containing not only glycerin as a substrate but also any by-product. Accordingly, by using 3-HPA of the present invention, a high purity of 3-HPA can be obtained by a simple procedures to separate or remove microbial cell/treated microbial cell. Further, by hydrogenating this 3-HPA, 1,3-propanediol can also be produced in a high purity.

In the present invention, separation/removal of microbial cell is not especially limited, and performed by a conventional method. Specifically, by using a known method such as filtration, ultrafiltration and settling, immobilized microbial cell can be easily separated from 3-HPA.

In the present invention, hydrogenation may be performed by any of fermentation or chemical synthesis method, but chemical synthesis method is preferable. This is because there is an advantage that by producing 1,3-propanediol from 3-HPA using chemical synthesis method, purity and yield of desired 1,3-propanediol can be improved. In this case, in the chemical synthesis method, conventional hydrogenation method may be used, and the reaction may be carried out either in a gas phase or a liquid phase. 3-HPA is hydrogenated preferably in a liquid phase, and more preferably in an aqueous solution, to form desired 1,3-propanediol. The chemical synthesis method to produce 1,3-propanediol from 3-HPA is not especially limited, and conventional methods can be used. For example, the methods include, for example, a method which comprises adding palladium carbon to 3-HPA, replacing a gas phase part with hydrogen, and hydrogenating the 3-HPA with hydrogen while stirring; a method which comprises hydrogenating 3-HPA in the presence of a heterogeneous catalyst having ruthenium supported on an oxide carrier at a temperature of 30 to 180° C. and under a hydrogen pressure of 5 to 300 bar, in an aqueous solution of pH value 2.5 to 7.0 (for example, JP-A-2002-516614); a method which comprises hydrogenating 5 to 100% by weight of 3-HPA in an aqueous solution, on a catalyst having platinum supported on titanium oxide as a carrier, at a temperature of 30 to 180° C., at pH value of 2.5 to 6.5, and under a hydrogen pressure of 5 to 300 bar (for example, JP-A-5-213800); and a method which comprises catalytically hydrogenating 3-HPA in an aqueous solution in the presence of a catalyst such as a catalyst having Pt supported on TiO₂ and a Ni/Al₂O₃/SiO₂-catalyst, under a hydrogen pressure of 5 to 300 bar, at pH value of 2.5 to 6.5, and at a temperature of 30 to 180° C. (for example, JP-A-6-40973).

According to the method of the present invention, 1,3-propanediol and water are mainly formed, and other by-products and secondary products are scarcely present. Hence, purification consists mainly of removal of water only, not complicated, and when fiber is produced using this 1,3-propanediol as a raw material thereafter, there is no risk resulting discoloration and defective polymerization in a product such as fiber. In this case, purification method of 1,3-propanediol to be used is not especially limited, and conventional methods can be used. For example, a method such as by distillation and reverse osmosis membrane can be used.

According to the present invention, by oxidizing 3-hydroxypropionaldehyde obtained similarly as above, 3-hydroxypropionic acid is produced. Thus, a third aspect of the present invention relates to a method which comprises a step of oxidizing the 3-hydroxypropionaldehyde produced by the method of the present invention to produce 3-hydroxypropionic acid.

In the present invention, in view of purity of 3-hydroxypropionic acid, microbial cell/treated microbial cell has been preferably separated or removed in advance prior to oxidation of 3-hydroxypropionaldehyde. In this case, separation/removal of microbial cell/treated microbial cell is not especially limited, but may be performed by conventional methods. Specifically, by using known methods such as filtration, ultrafiltration and settling, immobilized microbial cell can be removed from 3-HPA.

In the present invention, although production of 3-hydroxypropionic acid from 3-HPA may be performed by any of fermentation or chemical synthesis method, in view of purity and yield of 3-hydroxypropionic acid to be produced, chemical synthesis method is preferably used. In the oxidation according to the present invention, conventional oxidation methods can be used, and the reaction may be carried out either in a gas phase or a liquid phase. 3-HPA is oxidized preferably in a liquid phase, and more preferably in an aqueous solution. Method to be used in this case is not especially limited, conventional methods such as a method using platinum carbon, palladium carbon, or the like can be used. Specifically, the method includes a method which comprises adding palladium carbon to 3-HPA, and replacing a gas phase part with hydrogen, and oxidizing the 3-HPA with oxygen while stirring; and a method which comprises adding platinum carbon and sodium bicarbonate as catalysts to 3-HPA reaction liquid and contacting the 3-HPA with oxygen to oxidize the 3-HPA.

According to the method of the present invention, 3-hydroxypropionic acid can be mainly formed. For this reason, purification is easy. In this case, purification is not necessarily required. Purification method for 3-hydroxypropionic acid before use is not especially limited, and conventional methods, for example, a method such as distillation and reverse osmosis membrane can be used.

Further, according to the present invention, through a reaction of 3-hydroxypropionaldehyde obtained similarly as described above under acidic conditions, acrolein can be produced. Thus, a fourth aspect of the present invention relates to a method for producing acrolein which comprises a step of reacting the 3-hydroxypropionaldehyde produced by the method of the present invention under acidic conditions, to produce acrolein. Further, according to the present invention, through an oxidation of acrolein obtained by the fourth aspect of the present invention, acrylic acid is produced. Thus, a fifth aspect of the present invention relates to a method for producing acrylic acid which comprises a step of oxidizing the acrolein produced by the method of the present invention to produce acrylic acid.

In the fourth and fifth aspects of the present invention, in view of purities of acrolein and acrylic acid as final products, microbial cell/treated microbial cell has been preferably separated or removed in advance prior to oxidation of 3-hydroxypropionaldehyde. In this case, separation/removal of microbial cell/treated microbial cell is not especially limited, but may be performed by conventional methods. Specifically, by using known methods such as filtration, ultrafiltration and settling, immobilized microbial cell can be removed from 3-HPA.

In the present invention, production of acrolein from 3-HPA can be easily attained through a reaction of 3-HPA under acidic conditions. For example, acrolein can be efficiently produced by adding/mixing an acid such as hydrochloric acid, sulfuric acid and nitric acid, preferably hydrochloric acid, to a solution containing 3-HPA so as to give a pH value of 1 to 5, preferably 1.5 to 4.5, then standing at 5 to 90° C., preferably at 7 to 85° C. for 1 to 360 minutes, preferably 2 to 120 minutes. Since no components other than acid are used in this process, by-product is scarcely formed. Since the reaction has a high conversion ratio and a high selectivity, acrolein can be produced in a high purity and in a high yield.

In the present invention, acrolein thus formed is oxidized to produce acrylic acid. Production method from acrolein to acrylic acid is not especially limited, and may be any of fermentation or chemical synthesis method. However, in view of purity and yield of acrylic acid to be produced, chemical synthesis method is preferably used. In the oxidation according to the present invention, conventional oxidation methods can be used, and the reaction may be carried out either in a gas phase or a liquid phase. Further, oxidation method for acrolein in liquid phase is not also especially limited, and conventional methods can be used. For example, a method for producing acrylic acid by oxidizing acrolein in the presence of a palladium carbon catalyst while oxygen is added to reaction liquid containing acrolein, can be used. Further, oxidation method for acrolein in gas phase is not especially limited, and conventional methods can be used. For example, known methods such as disclosed in JP-A-64-63543 and JP-A-63-146841 can be used. Specifically, catalyst to be used for oxidizing acrolein to acrylic acid is not especially limited, and conventional catalysts can be used alone or in combination thereof. For example, catalysts containing molybdenum and vanadium, and preferably a catalyst represented by the general formula: Moₐ—V_{b}—W_{c}—Cu_{d}-Aₑ-B_{f}—C_{g}—Oₓ (wherein Mo represents molybdenum, V represents vanadium, W represents tungsten, Cu represents copper, A represents at least one element selected among antimony, bismuth, tin, niobium, cobalt, iron, nickel and chromium, B represents at least one element selected among alkali metals and alkaline earth metals, C represents at least one element selected from silicon, aluminum, zirconium and titanium, O represents oxygen, each of a, b, c, d, e, f, g and x represents atomic ratio of Mo, V, W, Cu, A, B, C and O, respectively, provided that when a=12, then b=2 to 14, c=0 to 12, d=0.1 to 5, e=0 to 5, f=0 to 5, g=0 to 20, and x is a value depending on oxidation state of each element) may be cited. Further, preparation method and mixing and molding method of catalyst to be used in this case are also not especially limited, and methods and raw materials which have been generally used in the art can be employed. Further, form of catalyst is also not especially limited, and for example, spherical, cylindrical and column-shaped forms can be used. As for forming method, support forming, extrusion forming, tablet compression, and the like can be used, and still further, a catalyst having these catalyst materials supported on a refractory carrier is also useful.

Further, reaction conditions used in the conversion from acrolein to acrylic acid is also not especially limited. For example, the reaction is performed by mixing acrolein with oxygen needed for conversion to acrylic acid and steam, and feeding this acrolein-containing gas under a reaction pressure in a range of atmospheric to 0.5 MPa, at a space velocity in a range of 300 to 5,000 hr⁻¹ (STP), with a reaction temperature being controlled at 200 to 400° C., and preferably 220 to 380° C.

The acrylic acid thus obtained is recovered by a common method. For example, the acrylic acid forming gas is, after cooled with a heat exchanger, made in countercurrent contact with a catching solvent containing a polymerization inhibitor to obtain acrylic acid aqueous solution, from which acrylic acid is isolated by a method such as extraction, distillation and azeotropic distillation.

Further, in the present invention, acrolein obtained in the fourth aspect is then converted to acrylic esters via oxidative esterification in the presence of a catalyst. Thus, a sixth aspect of the present invention relates to a method for producing an acrylic ester which comprises a step of subjecting the acrolein produced by the method of the present invention, to produce an acrylic ester. In this case, production method of an acrylic ester from acrolein is not especially limited, and any of fermentation or chemical synthesis method can be used. However, in view of purity and yield of an acrylic ester to be produced, chemical synthesis method is preferably used. In the oxidation according to the present invention, conventional oxidation methods can be used, and the reaction may be carried out either in a gas phase or a liquid phase. The reaction is performed particularly preferably in a liquid phase in the presence of a catalyst. In this case, as a catalyst, the similar catalyst as used in the production of acrylic acid can be used. Further, conditions of reaction from acrolein to acrylic ester is also not especially limited, and for example, the similar conditions as used in the production of acrylic acid can be used. The thus obtained acrylic ester is recovered by a common method.

### EXAMPLES

The present invention will be explained more specifically with referring to working examples below.

### Example 1

A strain JM109/vector 1 (DD)/vector 2 (DDR), which was obtained by transforming E. coli JM 109 as a host with vector 1 (FIG. 1A, SEQ ID NO: 1) obtained by inserting a gene coding for diol dehydratase of Klebsiella pneumoniae (ATCC 25955) into a plasmid having a replication origin (ori) derived from pBR322 and vector 2 (FIG. 1B, SEQ ID NO: 2) obtained by inserting a gene coding for diol dehydratase reactivating factor of Klebsiella pneumoniae (ATCC 25955) into a plasmid having a replication origin (ori) derived from p15A, was inoculated into a LB medium containing 50 μg/ml of ampicillin and 100 μg/ml of chloramphenicol, and cultured at 37° C. for 15 hours. The cultured liquid was inoculated into 200 ml of a LB medium containing 50 μg/ml of ampicillin and 100 μg/ml of chloramphenicol, and cultured with shaking at 37° C. When an absorbance at 660 nm reached 0.8 (OD=0.8) after initiation of the culture, IPTG was added so as to give a concentration of 1 mM, and cultured for further 5 hours, then the culture was stopped. Microbial cells were collected by centrifugation. Collected cells were washed twice with 50 mM of potassium phosphate buffer (pH 8), and suspended in 50 mM potassium phosphate buffer (pH 8) so as to give OD660=0.2. This microbial cell suspending fluid was used as a crude enzyme fluid. SEQ ID NO: 1 and SEQ ID NO: 2 show base sequences of vector 1 and vector 2 used in the present Example.

To 4 ml of the crude enzyme fluid, 3 ml of 50 mM potassium phosphate buffer (pH 8), 1 ml of 2 M glycerin, 1 ml of 0.5 M KCl and 1 ml of 150 μM coenzyme B12 were added, and the mixture was reacted at 37° C. for 60 minutes. An aliquot of the reaction liquid was taken and this aliquot is tested for quantitative determination of 3-hydroxypropionaldehyde therein. An equivalent amount of 0.1 M potassium citrate buffer (pH 3.0) was added to the reaction liquid to stop the reaction. An equivalent amount of water was added thereto, and 0.5 times by volume of an aqueous solution of 0.1% 3-methyl-2-benzothiazolinon hydrazone hydrochloride monohydrate was added, absorbance at 305 nm was measured to determine a concentration of 3-hydroxypropionaldehyde. The result indicated that 0.1 M 3-hydroxypropionaldehyde was formed in the reaction mixture. The residual reaction mixture was filtered to remove microbial cells. After the filtrate was placed into a test tube with a sealed cap, 0.015 g of 5% palladium carbon was added thereto, and a gas phase part was replaced with hydrogen gas. A balloon was filled with 500 ml of hydrogen gas under normal pressure, connected to the gas phase part and sealed, and the reaction was carried out with stirring at 60° C. for 5 hours in a hot-water bath. Analysis of the reaction mixture indicated that 0.098 M of 1,3-propandiol was formed. In this case, catalyst amount of microbial cells [X (U/g glycerin)], concentration of glycerin [Y (g/100 ml)], and X/Y² are summarized in Table 1 below. In Table 1, the values of [X (U/g glycerin)], [Y (g/100 ml)], and X/Y² disclosed in the documents listed as related references in the section of Description of Related Art are also summarized together in Table 1 below.

### Example 2

A strain JM109/vector 1′ (GD)/vector 2′ (GDR), which was obtained by transforming E. coli JM 109 as a host with vector 1′ (FIG. 2A, SEQ ID NO: 3) obtained by inserting a gene coding for glycerol dehydratase of Klebsiella pneumoniae (ATCC 25955) into a plasmid having replication origin (ori) derived from pBR322 and vector 2′ (FIG. 2B, SEQ ID NO: 4) obtained by inserting a gene coding for glycerol dehydratase reactivating factor of Klebsiella pneumoniae (ATCC 25955) into a plasmid having replication origin (ori) derived from p15A, was inoculated into a LB medium containing 50 μg/ml of ampicillin and 100 μg/ml of chloramphenicol, and cultured at 37° C. for 15 hours. The cultured liquid was inoculated into 200 ml of a LB medium containing 50 μg/ml of ampicillin and 100 μg/ml of chloramphenicol, and cultured with shaking at 37° C. When an absorbance at 660 nm reached 0.8 (OD=0.8) after initiation of the culture, IPTG was added so as to give a concentration of 1 mM, and cultured for further 5 hours, then the culture was stopped. Microbial cells were collected by centrifugation. Collected cells were washed twice with 50 mM of potassium phosphate buffer (pH 8), and suspended in 50 mM potassium phosphate buffer (pH 8) so as to give OD660=0.2. This microbial cell suspending fluid was used as a crude enzyme fluid. Toluene was added to the suspension so as to give a final concentration of 1%. After the mixture was stirred by using a vortex mixer for 5 minutes, microbial cells were collected by centrifugation. Microbial cells were suspended into 50 mM potassium phosphate buffer (pH 8) so as to give OD660 of 0.2. This suspension was designated as a toluene-treated microbial cell fluid. In this connection, SEQ ID NO: 3 and SEQ ID NO: 4 show base sequences of vector 1′ and vector 2′ used in the present Example.

To the toluene-treated microbial cell fluid, 3 ml of 50 mM potassium phosphate buffer (pH 8), 1 ml of 2M glycerin, 1 ml of 0.5 M KCl and 1 ml of 150 μM coenzyme B12 were added, and the mixture was reacted at 37° C. for 20 minutes. Analiquot of the reaction liquid was taken and this aliquot is tested for quantitative determination of 3-hydroxypropionaldehyde therein. An equivalent amount of 0.1 M potassium citrate buffer (pH 3.0) was added to the reaction liquid to stop the reaction. An equivalent amount of water was added thereto, and 0.5 times by volume of an aqueous solution of 0.1% 3-methyl-2-benzothiazolinon hydrazone hydrochloride monohydrate was added, absorbance at 305 nm was measured to determine a concentration of 3-hydroxypropionaldehyde. As a result, it was confirmed that 0.196 M of 3-hydroxypropionaldehyde was formed in the reaction mixture. A conversion ratio of glycerin to 3-hydroxypropionaldehyde was 98%. The residual reaction mixture was filtered to remove toluene-treated microbial cells. After the filtrate was placed into a test tube with a sealed cap, 0.015 g of 5% palladium carbon was added thereto, and a gas phase part was replaced with hydrogen gas. A balloon was filled with 500 ml of hydrogen gas under normal pressure, connected to the gas phase part and sealed, and the reaction was carried out with stirring at 60° C. for 5 hours in a hot-water bath. Analysis of the reaction mixture indicated that 0.196 M of 1,3-propandiol was formed. In this case, catalyst amount of microbial cells [X (U/g glycerin)], concentration of glycerin [Y (g/100 ml)], and X/Y² are shown in Table 1 below.

### Example 3

In the same manner as in Example 2, 0.196 M of 3-hydroxypropionaldehyde (conversion ratio of glycerin: 98%) was produced. The reaction mixture containing the same was adjusted to pH 2 with 35% hydrochloric acid, and allowed to stand at room temperature for 1 hour. The acrolein formed in the reaction mixture was quantitatively determined, to find that 0.130 M of acrolein was formed.

### Example 4

In the same manner as in Example 1, 0.188 M of 3-hydroxypropionaldehyde (conversion ratio of glycerin: 94%) was produced. After the reaction mixture was placed into a test tube with a sealed cap, 0.015 g of 5% palladium carbon was added thereto, and a gas phase part was replaced with oxygen gas. A balloon was filled with 1 litter of oxygen gas under normal pressure, connected to the gas phase part and sealed, and the reaction was carried out with stirring at 60° C. for 5 hours. Analysis of the reaction mixture indicated that 0.150 M of 3-hydroxypropionic acid was formed.

### Example 5

In the same manner as in Example 2, 0.196 M of 3-hydroxypropionaldehyde (conversion ratio of glycerin: 98%) was produced. 10 ml of the reaction mixture containing the same was adjusted to pH 2 with 35% hydrochloric acid, and allowed to stand at room temperature for 1 hour. The acrolein formed in the reaction mixture was quantitatively determined, to find that 0.150 M of acrolein was formed.

Subsequently, methanol was added to the resultant acrolein. The reaction was performed with stirring thoroughly under an oxygen atmosphere using 0.015 g of 5% palladium carbon as an oxidation catalyst, to form 0.188 M of methyl acrylate.

### Example 6

In the same manner as in Example 2, 0.196 M of 3-hydroxypropionaldehyde (conversion ratio of glycerin: 98%) was produced. The reaction mixture containing the same was adjusted to pH 2 with 35% hydrochloric acid, and allowed to stand at room temperature for 1 hour. The acrolein formed in the reaction mixture was quantitatively determined, to find that 0.148 M of acrolein (conversion ratio to 3-hydroxypropionaldehyde: 96.9%) was formed.

Subsequently, after the reaction mixture containing the resultant acrolein was placed into a test tube with a sealed cap, 0.015 g of 5% palladium carbon was added thereto, and a gas phase part was replaced with oxygen gas. A balloon was filled with 1 litter of oxygen gas under normal pressure, connected to the gas phase part and sealed, and the reaction was carried out with stirring at 60° C. for 5 hours. Analysis of the reaction mixture indicated that 0.120 M of acrylic acid was formed.

### Example 7

Klebsiella pneumoniae (ATCC 25955) was anaerobically cultured in a medium containing glycerin as a carbon source to grow until reaching a logarithmic growth phase. The microbial cell culture at this stage was treated with 1% toluene in the same manner as described in Example 2, and the treated microbial cells were collected. The collected microbial cells of 20 g (wet weight, enzymatic activity 4000 U) of Klebsiella pneumoniae (ATCC 25955) were added to l litter of 50 mM potassium phosphate buffer (pH 8) containing 135 μM coenzyme B12 and 0.2 M glycerin, and the resultant mixture was reacted at 37° C. for 120 minutes. After the reaction mixture was filtered to remove off the toluene-treated microbial cells, an aliquot thereof was taken out and an amount of 3-hydroxypropionaldehyde therefor was quantitatively determined to find that 0.196 M of 3-hydroxypropionaldehyde was formed. The resultant 3-hydroxypropionaldehyde was placed into a 1 litter of separable flask, 1.5 g of 5% palladium carbonawas added thereto, and a gas phase part was replaced with oxygen. A small amount of oxygen was passed through the gas phase part in order to prevent the penetration of outside gas. The mixture was reacted at 60° C. for 5 hours with stirring. The reaction mixture was analyzed to detect 0.178 M of 3-hydroxypropionic acid. Catalyst amount of the toluene-treated microbial cells [X (U/g glycerin)], concentration of glycerin [Y (g/100 ml)], and X/Y² are summarized in Table 1 below.

### Example 8

Klebsiella pneumoniae (ATCC 25955) was anaerobically cultured in a medium containing glycerin as a carbon source to grow until reaching a logarithmic growth phase. The microbial cell culture at this stage was treated with 1% toluene in the same manner as described in Example 2, and the treated microbial cells were collected. The collected microbial cells of 20 g (wet weight, enzymatic activity 4000 U) of Klebsiella pneumoniae (ATCC 25955) were added to 1 litter of 50 mM potassium phosphate buffer (pH 8) containing 135 μM coenzyme B12 and 0.2 M glycerin, and the resultant mixture was reacted at 37° C. for 120 minutes. After the reaction mixture was filtered to remove off the toluene-treated microbial cells, an aliquot thereof was taken out and an amount of 3-hydroxypropionaldehyde therefor was quantitatively determined to find that 0.197 M of 3-hydroxypropionaldehyde was formed. The resultant reaction mixture containing 3-hydroxypropionaldehyde was adjusted to pH 2 with 35% hydrochloric acid, and allowed to stand at room temperature for 1 hour. An amount of acrolein formed in the reaction mixture was quantitatively determined, to find that 0.130 M of acrolein was formed. Catalyst amount of the toluene-treated microbial cells (X (U/g glycerin)), concentration of glycerin (Y (g/100 ml)), and X/Y² are summarized in Table 1 below.

### [Table 1]

TABLE 1

| | Catalytic | Substrate | | |
|---|---|---|---|---|
| | amount X | conc. Y | | Conversion |
| Source | (U/g Gly) | (%) | X/Y² | (%) |
| J. Bac. Vol. 181, No. 13, | 8 | 0.92 | 8.989 | 1.4 |
| '99, p. 4110-4113 | | | | |
| The J. of Biolog. Chem., | 1 | 1.84 | 0.273 | 0.17 |
| Vol. 272, No. 51, '97 | | | | |
| Arch. Microbiol., 174, | 8 | 0.92 | 8.989 | 1.4 |
| 81-88 (2000) | | | | |
| The J. of Biolog. Chem., | 1 | 11.04 | 0.010 | 0.27 |
| Vol. 274, No. 6, '99 | | | | |
| The J. of Biolog. Chem., | 7 | 13.8 | 0.034 | 0.75 |
| Vol. 274, No. 6, '99 | | | | |
| Example 1 | 91 | 1.84 | 27 | 50 |
| Example 2 | 533 | 1.84 | 157 | 98 |
| Example 3 | 533 | 1.84 | 157 | 98 |
| Example 7 | 217 | 1.84 | 64 | 98 |
| Example 8 | 217 | 1.84 | 64 | 98.5 |

### Example 9

To 10 ml of 50 mM potassium phosphate buffer (pH 8) containing 4.6% (0.5 M) of glycerin as a substrate and 135 μM of coenzyme B12, an enzymatic activity 200 U/g wet weight of toluene-treated (Klebsiella pneumoniae) microbial cells were added so as to give a catalyst amount as shown in Table 2 below. The mixture was reacted with stirring at 37° C. for 6 hours in dark. The toluene-treated microbial cells were removed off by centrifugation of the reaction mixture after performing the reaction for a prescribed time. Then, the conversion ratio from glycerin to 3-hydroxypropionaldehyde was determined using the supernatant. Catalyst amount of the toluene-treated microbial cells [X (U/g glycerin)], concentration of glycerin [Y (g/100 ml)], and X/Y² are summarized in Table 2 below.

### [Table 2]

TABLE 2

| Amount of | Catalytic | Substrate | | |
|---|---|---|---|---|
| microbial | amount X | conc. Y | | Conversion |
| cells (g) | (U/g glycerin) | (%) | X/Y² | (%) |
| 0.1 | 920 | 4.6 | 43 | 52 |
| 0.2 | 1840 | 4.6 | 87 | 89 |
| 0.3 | 1304 | 4.6 | 95 | 96 |
| 0.5 | 2174 | 4.6 | 103 | 98 |
| 1 | 4348 | 4.6 | 205 | 98 |
| 2 | 8696 | 4.6 | 411 | 98 |
| 5 | 21739 | 4.6 | 1027 | 97 |
| 10 | 43478 | 4.6 | 2055 | 98 |
| 15 | 65217 | 4.6 | 3082 | 95 |
| 20 | 86957 | 4.6 | 4109 | 95 |
| 25 | 108696 | 4.6 | 5137 | 90 |
| 30 | 130435 | 4.6 | 6164 | 85 |
| 35 | 152174 | 4.6 | 7192 | 70 |
| 40 | 173913 | 4.6 | 8219 | 65 |
| 45 | 195652 | 4.6 | 9146 | 51 |
| 50 | 217391 | 4.6 | 10274 | 30 |

As shown in the results of the Table 2, when X/Y² exceeds 80, a high conversion ratio as of about not less than 90% can be achieved. When the value is not more than 8219, it is shown that the conversion ratio is also below 70%. Further, when X/Y² is within a range of 87 to 6164, a conversion ratio of not less than 80% can be achieved, especially when X/Y² is in a range of 95 to 5137, it is shown that an extremely high conversion ratio not less than 80% can be achieved.

### Example 10

A toluene-treated microbial cell culture of a strain JM 109/vector 1′ (GD) was prepared by the same way as described in Example 2. The toluene-treated microbial cells (enzymatic activity 9, 900 U/g wet weight) was added into 50 mM potassium phosphate buffer (pH 8) containing 9.2% (1 M) of glycerin as a substrate and 135 μM of coenzyme B12 so as to give a catalytic amount as shown in Table 3 below, and the total volume was adjusted to 10 ml. The mixture was reacted at 37° C. for 2 hours in dark. The toluene-treated microbial cells were removed off by centrifugation of the reaction mixture after performing the reaction for a prescribed time, and a conversion ratio from glycerin to 3-hydroxypropionaldehyde was determined using the supernatant. Catalyst amount of the toluene-treated microbial cells [X (U/g glycerin)], concentration of glycerin [Y (g/100 ml)], and X/Y² are summarized in Table 3 below.

### [Table 3]

TABLE 3

| Amount of | Catalytic | Substrate | | |
|---|---|---|---|---|
| microbial | amount X | conc. (Y) | | Conversion |
| cells (g) | (U/g glycerin) | (%) | X/Y² | (%) |
| 0.051 | 543 | 9.2 | 6 | 43.8 |
| 0.101 | 1087 | 9.2 | 13 | 72.1 |
| 0.202 | 2174 | 9.2 | 26 | 79.3 |
| 0.303 | 3261 | 9.2 | 39 | 79.4 |
| 0.404 | 4348 | 9.2 | 51 | 83.3 |
| 0.752 | 8096 | 9.2 | 77 | 78.3 |

As shown in the result of the Table 3, when X/Y² exceeds 10, a high conversion ratio as of not less than 70% can be achieved. Especially when X/Y² exceeds 50, it is shown that an extremely high Conversion ratio as of not less than 80% can be achieved. Further, it is noted in this Example that even if a considerably high concentration of substrate as high as 9.2% is used, an conversion ratio of not less than 70% can be achieved by applying an action of the toluene-treated microbial cells to glycerin under conditions that X/Y² becomes not less than 10. In addition, as shown in this Example, according to the method of the present invention, since a high conversion ratio can be achieved even in a high concentration of substrate, it is suggested that the method of the present invention is very advantageous from the viewpoint of an industrial level.

### INDUSTRIAL APPLICABILITY

According to the method for producing 3-hydroxypropionaldehyde of the present invention, by acting diol/glycerol dehydratase and/or diol/glycerol dehydratase reactivating factor on 3-HPA while an amount of enzyme being controlled within a specified range, the reaction from glycerin to 3-HPA occurs selectively without inducing any side reaction other than the reaction from glycerin to 3-HPA, such a high conversion ratio as not less than 80%, in some cases, not less than 90% can be attained, and 3-hydroxypropionaldehyde can be produced in a high yield. Further, in the method of the present invention, since the reaction from glycerin to 3-HPA can be performed without using a fermentation method, 3-hydroxypropionaldehyde can be produced with a high purity scarcely containing by-product. Accordingly, 3-HPA obtained by the method of the present invention can be used as an intermediate in producing 1,3-propanediol by hydrogenation, through a very simple purification process for separation of microbial cell/treated microbial cell such as filtration, ultrafiltration and settling, and the like. In addition to the advantage, since only 1,3-propanediol and water mainly remain after completion of the reaction, and no organic solvents are needed for purification, the method does not require recovery of organic solvent and post-treatment, which is preferable from the viewpoint of environment.

Further, the present invention relates to a method for obtaining acrolein by reacting the 3-hydroxypropionaldehyde scarcely containing by-product as described above under acidic conditions, and further for producing acrylic acid by oxidizing the acrolein; and a method for producing an acrylic ester by reacting 3-hydroxypropionaldehyde scarcely containing by-product as described above under acidic conditions to obtain acrolein, and further subjecting the acrolein to oxidative esterification. According to the method, since 3-hydroxypropionaldehyde as a raw material scarcely contains by-product, acrolein produced using this, and further acrylic acid and an acrylic ester produced from this have also high purities.

According to the present invention, since 3-HPA can be produced in a high conversion ratio and a high purity ratio, and scarcely containing by-product, 1,3-propanediol, 3-hydroxypropionic acid, acrolein, acrylic acid and acrylic ester can be produced in high yields and with high purities, respectively. Accordingly, 1,3-propanediol thus produced can be used as a monomer to be used for producing polyesters and polyurethanes, and as a starting material for synthesis of cyclic compounds. Further, fibers produced by using this compound do not show discoloration because 1,3-propanediol scarcely contains by-product. Also, acrylic acid/acrylic ester thus produced can be used not only for copolymers for acrylic fibers or for adhesives/agglutinant as an emulsion, but also for coating materials, textile processing, leathers, construction materials, and the like.

### BRIEF DESCRIPTION OF THE DRAWINGS

FIG. 1A shows a gene map of vector 1, which is an expressing plasmid used in Example 1. In FIG. 1A, Amp^{r} represents an ampicillin-resistant gene; Cm^{r} represents a chloramphenicol-resistant gene; lacI_{q} represents a lactose repressor gene; and P_{tac} represents a tac promoter, respectively.

FIG. 1B shows a gene map of vector 2, which is an expressing plasmid used in Example 1. In FIG. 1B, Amp^{r} represents an ampicillin-resistant gene; Cm^{r} represents a chloramphenicol-resistant gene; lacI_{q} represents a lactose repressor gene; and P_{tac} represents a tac promoter, respectively.

FIG. 1B shows a gene map of vector 1′, which is an expressing plasmid used in Example 2. In FIG. 2A, Amp^{r} represents an ampicillin-resistant gene; Cm^{r} represents a chloramphenicol-resistant gene; lacI_{q} represents a lactose repressor gene; and P_{tac} represents a tac promoter, respectively.

FIG. 1B shows a gene map of vector 2′, which is an expressing plasmid used in Example 2. In FIG. 2A, Amp^{r} represents an ampicillin-resistant gene; Cm^{r} represents a chloramphenicol-resistant gene; lacI_{q} represents a lactose repressor gene; and P_{tac} represents a tac promoter, respectively.

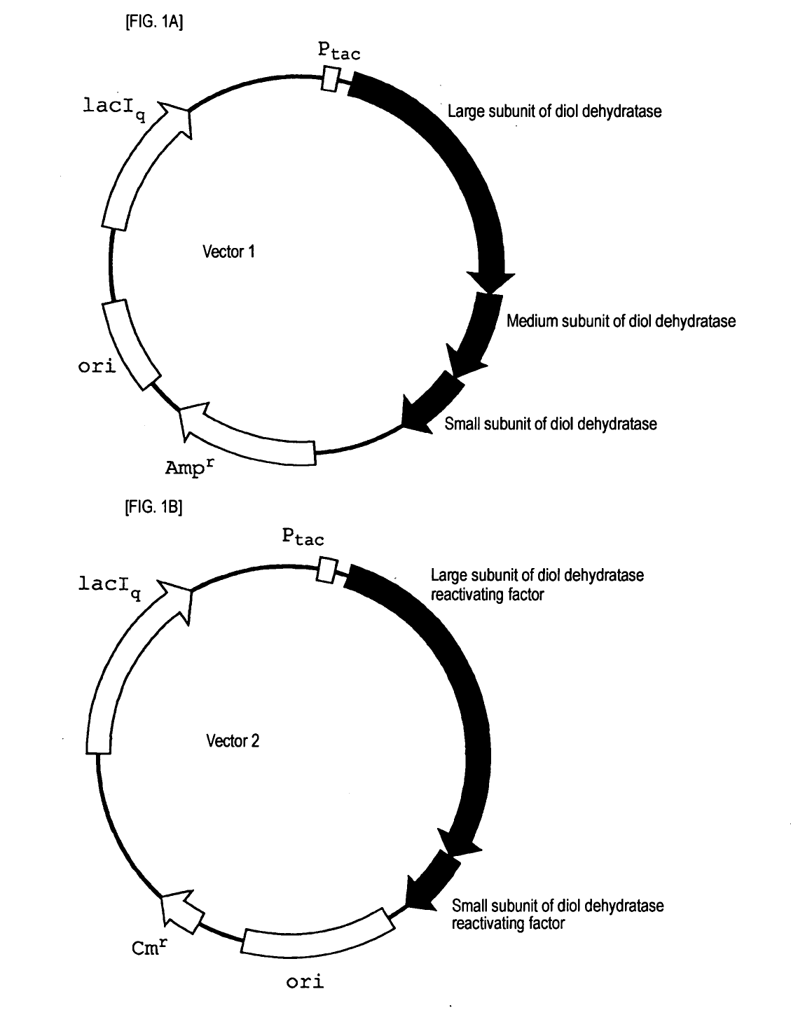

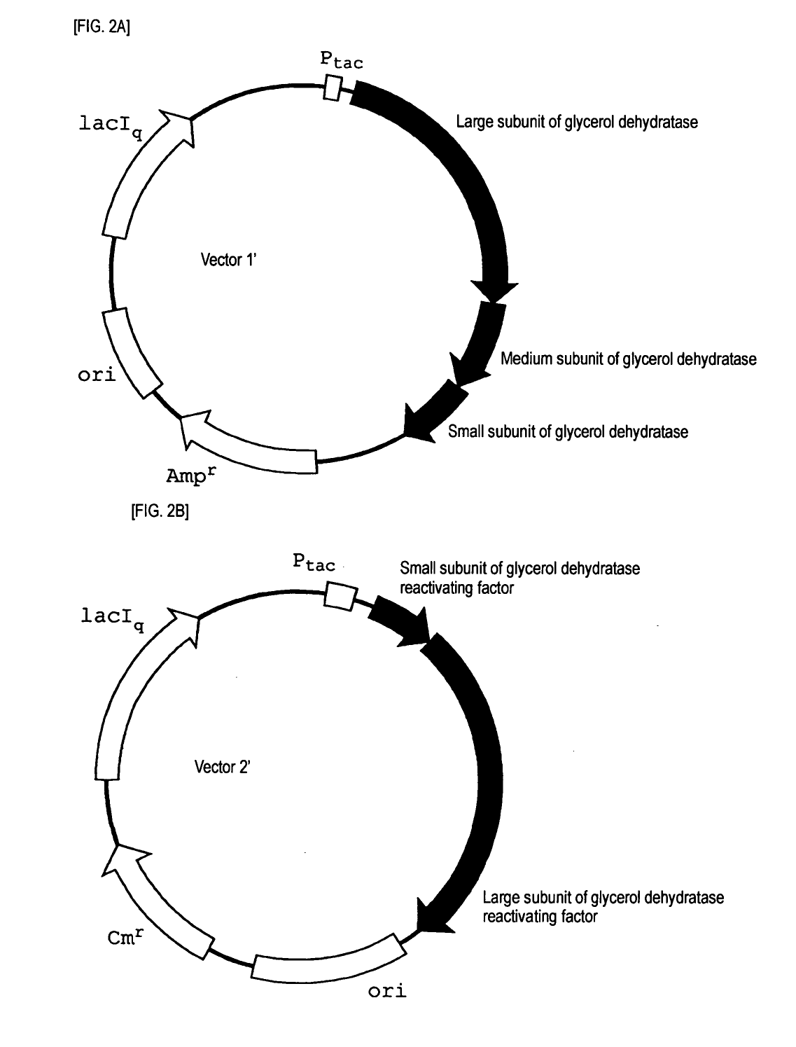

1. A method for producing 3-hydroxypropionaldehyde which comprises a step of dehydrating glycerin using a microbial cell and/or a treated microbial cell containing diol dehydratase and/or glycerol dehydratase, and optionally diol dehydratase reactivating factor and/or glycerol dehydratase reactivating factor, under conditions so as to give a value (X/Y²) calculated by dividing a catalytic amount [X (U/g glycerin)] of diol dehydratase and/or glycerol dehydratase by square of glycerin concentration [Y (g/100 ml)] within a range of 10 to 8,000, to produce 3-hydroxypropionaldehyde.

2. A method according to claim 1, wherein the dehydration of glycerin is performed using a microbial cell under aerobic conditions.

3. A method according to claim 1, wherein the dehydration of glycerin is performed using a treated microbial cell.

4. A method for producing 1,3-propanediol which comprises a step of removing the microbial cell and/or treated microbial cell from the 3-hydroxypropionaldehyde produced by the method set forth in claim 1, subsequently hydrogenating said 3-hydroxypropionaldehyde to produce 1,3-propanediol.

5. A method for producing 3-hydroxypropionic acid which comprises a step of oxidizing the 3-hydroxypropionaldehyde produced by the method set forth in claim 1 to produce 3-hydroxypropionic acid.

6. A method for producing acrolein which comprises a step of reacting the 3-hydroxypropionaldehyde produced by the method set forth in claim 1 under acidic conditions, to produce acrolein.

7. A method for producing acrylic acid which comprises a step of oxidizing the acrolein produced by the method set forth in claim 6 to produce acrylic acid.

8. A method for producing an acrylic ester which comprises a step of subjecting the acrolein produced by the method set forth in claim 6 to the oxidative esterification, to produce an acrylic ester.

9. A method for producing 1,3-propanediol which comprises a step of removing the microbial cell and/or treated microbial cell from the 3-hydroxypropionaldehyde produced by the method set forth in claim 2, subsequently hydrogenating said 3-hydroxypropionaldehyde to produce 1,3-propanediol.

10. A method for producing 1,3-propanediol which comprises a step of removing the microbial cell and/or treated microbial cell from the 3-hydroxypropionaldehyde produced by the method set forth in claim 3, subsequently hydrogenating said 3-hydroxypropionaldehyde to produce 1,3-propanediol.

11. A method for producing 3-hydroxypropionic acid which comprises a step of oxidizing the 3-hydroxypropionaldehyde produced by the method set forth in claim 2 to produce 3-hydroxypropionic acid.

12. A method for producing 3-hydroxypropionic acid which comprises a step of oxidizing the 3-hydroxypropionaldehyde produced by the method set forth in claim 3 to produce 3-hydroxypropionic acid.

13. A method for producing acrolein which comprises a step of reacting the 3-hydroxypropionaldehyde produced by the method set forth in claim 2 under acidic conditions, to produce acrolein.

14. A method for producing acrolein which comprises a step of reacting the 3-hydroxypropionaldehyde produced by the method set forth in claim 3 under acidic conditions, to produce acrolein.

15. A method for producing acrylic acid which comprises a step of oxidizing the acrolein produced by the method set forth in claim 13 to produce acrylic acid.

16. A method for producing acrylic acid which comprises a step of oxidizing the acrolein produced by the method set forth in claim 14 to produce acrylic acid.

17. A method for producing an acrylic ester which comprises a step of subjecting the acrolein produced by the method set forth in claim 13 to the oxidative esterification, to produce an acrylic ester.

18. A method for producing an acrylic ester which comprises a step of subjecting the acrolein produced by the method set forth in claim 14 to the oxidative esterification, to produce an acrylic ester.


SEQUENCE LISTING (4 sequences)

SEQ ID NO: 1 - DNA, 7183 bp, Artificial Sequence
  Description of Artificial Sequence Synthetic plasmid pBR322
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtacatatgagatcgaaaagatttgaagcactggcgaaacgccctgtgaatcaggacggcttcgttaaggagtggatcgaagaaggctttatcgcgatggaaagcccgaacgacccaaaaccgtcgattaaaatcgttaacggcgcggtgaccgagctggacgggaaaccggtaagcgattttgacctgatcgaccactttatcgcccgctacggtatcaacctgaaccgcgccgaagaagtgatggcgatggattcggtcaagctggccaacatgctgtgcgatccgaacgttaaacgcagcgaaatcgtcccgctgaccaccgcgatgacgccggcgaaaattgtcgaagtggtttcgcatatgaacgtcgtcgagatgatgatggcgatgcagaaaatgcgcgcccgccgcaccccgtcccagcaggcgcacgtcaccaacgtcaaagataacccggtacagattgccgccgacgccgccgaaggggcatggcgcggatttgacgaacaggaaaccaccgttgcggtagcgcgctatgcgccgttcaacgccatcgcgctgctggtgggctcgcaggtaggccgtccgggcgtgctgacgcagtgctcgctggaagaagccaccgagctgaagctcggcatgctgggccacacctgctacgccgaaaccatctccgtctacggcaccgagccggtctttaccgacggcgacgacacgccgtggtcgaagggcttcctcgcctcgtcctacgcctctcgcgggctgaaaatgcgctttacctccggctccggctcggaagtgcagatgggctacgccgaaggcaaatccatgctttatctggaagcgcgctgcatctacatcaccaaagccgcgggcgtacagggtctgcaaaacggttccgtaagctgcatcggcgtgccgtctgcggtgccttccggcattcgcgcggtgctggcggaaaacctgatctgttcgtcgctggatctggagtgcgcctccagcaacgaccagaccttcacccactccgatatgcgtcgtaccgcgcgcctgctgatgcagttcctgccgggcaccgactttatctcctccggttattccgcggtgccgaactacgacaacatgttcgccggctccaacgaagatgccgaagactttgacgactacaacgtcatccagcgcgacctgaaggtggacggcggtttgcgtccggttcgcgaagaggacgtcatcgccatccgtaacaaagccgcccgcgcgctgcaggccgtgtttgccggaatggggctgccgccgattaccgatgaagaagttgaagccgcgacctacgcccacggttcgaaagatatgccggagcgcaacatcgtcgaagacatcaagttcgcccaggaaatcatcaataaaaaccgcaacggtctggaagtggtgaaagcgctggcgcagggcggattcaccgacgtggcccaggacatgctcaacatccagaaagctaagctgaccggggactacctgcatacctccgcgattatcgtcggcgacgggcaggtgctgtcagccgtcaacgacgtcaacgactatgccggtccggcaacgggctatcgcctgcagggcgaacgctgggaagagattaaaaacatccctggcgctcttgatcccaacgagattgattaaggggtgagaaatggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttgataaaggataactccatgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaacttcattccgggcccgtcgacagatccccgggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 2 - DNA, 6607 bp, Artificial Sequence
  Description of Artificial Sequence Synthetic plasmid p15A
gaattaattctggcgaatcctctgaccagccagaaaacgacctttctgtggtgaaaccggatgctgcaattcagagcgccagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaagcttactccccatccccctgttgacaattaatcatcggctcgtataatgtgtggaattgtgagcggataacaatttcacacaggaaacaggatcctaggaggtttaaacatatgcgatatatagctggcattgatatcggcaactcatcgacggaagtcgccctggcgaccctggatgaggctggcgcgctgacgatcacccacagcgcgctggcggaaaccaccggaatcaaaggcacgttgcgtaacgtgttcgggattcaggaggcgctcgccctcgtcgccagaggcgccgggatcgccgtcagcgatatttcgctcatccgcatcaacgaagcgacgccggtgattggcgatgtggcgatggaaaccattaccgaaaccatcatcaccgaatcgaccatgatcggccataacccgaaaacgcccggcggcgcggggcttggcacaggcatcaccattacgccgcaggagctgctaacccgcccggcggacgcgccctatatcctggtggtgtcgtcggcgttcgattttgccgatatcgccagcgtgattaacgcttccctgcgcgccgggtatcagattaccggcgtcattttacagcgcgacgatggcgtgctggtcagcaaccggctggaaaaaccgctgccgatcgttgacgaagtgctgtacatcgaccgcattccgctggggatgctggcggcgattgaggtcgccgttccggggaaggtcatcgaaaccctctctaacccttacggcatcgccaccgtctttaacctcagccccgaggagacgaagaacatcgtcccgatggcccgggcgctgattggcaaccgttccgccgtggtggtcaaaacgccatccggcgacgtcaaagcgcgcgcgatacccgccggtaatcttgagctgctggcccagggccgtagcgtgcgcgtggatgtggccgccggcgccgaagccatcatgaaagcggtcgacggctgcggcaggctcgataacgtcaccggcgaatccggcaccaatatcggcggcatgctggaacacgtgcgccagaccatggccgagctgaccaacaagccgagcagcgaaatatttattcaggacctgctggccgttgatacctcggtaccggtgagcgttaccggcggtctggccggggagttctcgctggagcaggccgtgggcatcgcctcgatggtgaaatcggatcgcctgcagatggcaatgatcgcccgcgaaatcgagcagaagctcaatatcgacgtgcagatcggcggcgcagaggccgaagccgccatcctgggggcgctgaccacgccgggcaccacccgaccgctggcgatcctcgacctcggcgcgggctccaccgatgcctccatcatcaaccccaaaggcgacatcatcgccacccatctcgccggcgcaggcgacatggtgacgatgattattgcccgcgagctggggctggaagaccgctatctggcggaagagatcaagaagtacccgctggctaaggtggaaagcctgttccatttacgccacgaggacggcagcgtgcagttcttctccacgccgctgccgcccgccgtgttcgcccgcgtctgcgtggtgaaagcggacgaactggtgccgctgcccggcgatttagcgctggaaaaagtgcgcgccattcgccgcagcgccaaagagcgggtctttgtcaccaacgccctgcgcgcgctgcgtcaggtcagccccaccggcaacattcgcgatattccgttcgtggtgctggtcggcggttcgtcgctggatttcgaagtcccgcagctggtcaccgatgcgctggcgcactaccgcctggttgccggacggggaaatattcgcggcagcgagggcccccgaaacgcggtggccaccggcctgattctctcctggcataaggagtttgcgcatgaacggtaatcacagcgccccggccatcgcgatcgccgtcatcgacggctgcgacggcctgtggcgcgaagtgctgctgggtatcgaagaggaaggtatccctttccggctccagcatcacccggccggagaggtcgtggacagcgcctggcaggcggcgcgcagctcgccgctgctggtgggcatcgcctgcgaccgccatatgctggtcgtgcactacaagaatttacccgcatcggcgccgctttttacgctgatgcatcatcaggacagtcaggcccatcgcaacaccggtaataacgcggcacggctggtcaaggggatccctttccgggatctgaatagcgaagcaacaggagaacagcaggatgaataagatctcgggtagcccgcctaatgagcgggcttttttttatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccaaagcaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttg

SEQ ID NO: 3 - DNA, 7023 bp, Artificial Sequence
  Description of Artificial Sequence Synthetic plasmid pBR322
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtacatatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgataattccgggcccgtcgacagatccccgggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 4 - DNA, 6608 bp, Artificial Sequence
  Description of Artificial Sequence Synthetic plasmid p15A
gaattaattctggcgaatcctctgaccagccagaaaacgacctttctgtggtgaaaccggatgctgcaattcagagcgccagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaagcttactccccatccccctgttgacaattaatcatcggctcgtataatgtgtggaattgtgagcggataacaatttcacacaggaaacaggatcctaggaggtttaaacatatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaagatcctaggaggtttaaacatatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaagatctcgggtagcccgcctaatgagcgggcttttttttatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccaaagcaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttg